(19) United States
(12) Patent Application Publication
(10) Pub. No.: US 2021/0315910 A1
(43) Pub. Date: Oct. 14, 2021

(54) Method to mitigate morbidity and mortality in virally induced forms of ACE2 receptor pathology progressing to SARS or ARDS.

(71) Applicant: Vivi Robyn Stafford, Los Angeles, CA (US)

(72) Inventor: Vivi Robyn Stafford, Los Angeles, CA (US)

(21) Appl. No.: 16/842,787

(22) Filed: Apr. 8, 2020

(51) Int. Cl. A61K 31/65; A61K 31/585

(52) U.S. Cl. CPC A61K 31/65; A61K 45/06; A61K 31/585

(57) ABSTRACT

ACE receptors are affected in severe acute respiratory distress syndrome related coronaviruses. ACE genes are directly related to the morbidity and mortality of those with cystic fibrosis. The thick, sticky mucus in the respiratory, and digestive systems, is seen in the inherited disease cystic fibrosis. Viral induced sticky mucus in the respiratory and digestive systems can also be appreciated as a response to viral pathogens where the cycle of mucus production in vivo induces the recruitment of more mucus production to the extent that cellular damage occurs within the lower respiratory track requiring intubation as a life saving measure. In the most severe cases mechanical intubation fails due to the fact that no control over the recruitment of mucus production was achieved at the onset. SARS pathology shows inflammatory exudation in the alveoli and interstitial tissue, with hyperplasia of fibrous tissue and fibrosis. Inherited cystic fibrosis pathology shows atelectasis, mucoid impaction, acute and chronic inflammation, bronchiectasis, cyst formation, and fibrosis widespread. A virally induced disorder in relations to ACE2 receptors can be treated successfully at the early onset with inherited cystic fibrosis disease mimicking techniques with the efforts of minimizing the activity and utilization of the ACE2 receptor. Cystic fibrosis lung infections, and opportunistic pathogens contribute to chronic airway inflammation that is characterized by neutrophil/macrophage infiltration, cytokine release and ceramide accumulation. In terms of virally induced forms off ACE2 receptor pathologies, as it related to coronavirus such as Covid 19, presumably ceramide precursors which aid in intracellular transport of the virus into the cell via inflammation and remodeling is present in alveolar tissues of the lung in patients with cystic fibrosis while the same pathology occurs in patients with virally induced forms of ACE2 pathology which to often progresses to SARS or ARDS.

### BACKGROUND OF THE INVENTION

This invention was not made with U.S. government support.

Coronavirus has been well studied for decades in term of its propensity to cause disease in humans. Coronaviruses, specifically Covid 19, enters the body through the mouth; often from hands which the virus hitches a ride. Presumably, the virus is aided in hitching a ride into the mucous membranes of a human through superinfections or infections via bacteria symbiotic or pathological to humans. Frequently, Covid 19 transfers from the hands to the mouth through the touching of the face of infected individuals. COVID 19 is a contagious and infectious disease of the upper respiratory tract which causes societal catastrophe through extensive morbidity and mortality when it progresses to a lower respiratory tract or systemic illness. There currently is an approved treatment under emergency FDA approval only which is hydroxychloroquine and chloroquine. However, this treatment is controversial, is not currently proven scientifically to be effective with tremendous benefits vs risks ratios and has the risks for serious and irreversible toxicity, long term sequelae detrimental to one's health and could cause morbidity or death secondary to drug toxicity.

Covid19 is a virus considered new and likely of animal origin with cross transmission into human beings through a presumable intermediary where it had run amok worldwide causing massive hospitalizations and death; causing more than an economic and emotional burden on one of the most stable nations in the world. The illness in a confirmed percentage of the population causes a cytokine storm which may contribute to death from those infected with the virus. The illness is spread from individuals touching items, such as contaminated door knobs subsequently followed with contact to the face in relatively short amount of times. The virus may travel large distances as an aerosol, for example, when an infected individual coughs, sneezes or vocalizes. The virus makes its way into the throat from an environmental exposure via the mouth, tear troughs or nostrils, travels into the sinuses where it causes an acute sinusitis and then it drops from the septum and moves caudally into the respiratory tree where it then progresses to an unarguably life threatening illness. Throughout the upper respiratory track and respiratory tree, the virus searches out ACE 2 receptors and binds to the ACE 2 receptors causing a cough of a reflexive nature as the virus grabs unto these receptors in order to replicate or function at its optimal capacity. The ACE 2 receptor response may be marked where the patient may describe chest tightening as an intense as hundreds of pounds squeezing their chests. The disease may progress in some to a persistent state of inflammation which can cause death or long term disability where lab values of c reactive protein clearly establishes the inflammation. Viruses can be symbiotic partners in the health of their hosts. In this capacity, Covid 19 has the capacity to have a symbiotic relationship with a bacterium where it then may cause an aggressive bacterial or fungal superinfection in humans. In some cases, viruses have fused with their hosts in symbiogenetic relationships. Such a mutualistic interaction may cause grave presentations in those infected with Covid 19.

Cyclines are a family of antibiotics that act by inhibiting bacterial protein synthesis. Cyclines act by binding to several proteins in the 30S ribosomal small subunit and to different ribonucleic acids in the 16S ribosomal RNA. The 30S ribosomal small unit is implicated in the binding of transfer RNA to messenger RNA. Viruses replicate only inside the living cells of an organism. Viruses cannot reproduce independently and reproduce within a host cell. Some viruses replicate within a bacterial cell. In order for a virus to reproduce it requires a host and processes of attachments to specific receptors on the host cellular surface. The virus must penetrate to a specific receptor to induce entry into a cell. Furthermore, a viral capsid is removed and degraded by viral enzymes or host enzymes releasing the viral genomic nucleic acid. A virus must replicate, in which a process culminates in the de novo synthesis of viral proteins and genome. In terms of cytolytic viruses, after a virus replicates de novo synthesis of viral genome and proteins, the virion release results in the death of an infected host cell. A host cell for the virus can be a bacteria or it can be a human cell. Cytopathic viruses often do not kill the infected cell. Coronaviruses contain a single stranded, 5′-capped, positive strand RNA molecule that ranges from 26-32 kb and that contains at least 6 open reading frames. The first open reading frame comprises approximately two-thirds of the genome and encodes replicase proteins. Cyclins' disrupt the replicase proteins and stall the progression of the disease to end organ damage as seen in COVID 19. Inhibition of proliferation by doxycycline is beneficial. Induction of potential beneficial immune and metabolic pathways by metronidazole may be appreciated. In addition, in relations to cycline's affecting immune responses, persistent alterations in microRNA and mRNA expression profiles after the recovery period indicate that cyclines in conjunction with medications or independently induce long-term epigenetic modifications in both T-cell subsets and influences or disrupts other immunological pathways. Therefore, doxycyclines can be used for periods of time and then stopped with protective measures from Covid 19 or other coronaviruses progressing. Currently, no antiviral therapy has yet been proven universally useful for SARS. Attempts to test potential anti-SARS agents using antiviral antibodies, entry inhibitors, proteinase inhibitors, calpain inhibitors, ribavirin (nucleoside analogues), interferons, and short interfering RNAs were riddled with contradictory reports from different laboratories. Covid has a dependency on the host cell for spread onto the infected individual. Therefore, antibiotics are an effective method in the treatment through virus dependence on the concomitant invasion of bacterial cells, through direct effects on viral replication and through the inhibition of viral activity and behavior. Additionally, zinc inhibits Coronavirus RNA polymerase activity in vitro. Ionophores block replication of these viruses in cell culture. Cyclines act as ionophores which bind to divalent metal cations. The ionophoric ability of cyclines allows the passage of zinc and other trace minerals into the host cell inhibiting viral replication. Doxycycline specifically has anti-inflammatory properties which can be used uniquely in the mitigation of the detrimental SARS type illnesses which progresses in a significant portion of in those infected with Covid 19. Spironolactone has added benefits as it decreases ACE2 receptor availability to viral binding proteins. Metronidazole is also beneficial in inhibiting bacterial overgrowth. Doxycycline blocks the expression of parasitic types of genes including when a virus expresses itself as a parasite. Metabolic profile enhancement is beneficial to maximizing results.

### BRIEF DESCRIPTION OF THE INVENTION

The single therapy of Doxycycline independently will mitigate the infection of Covid 19 minimizing the risks of it progressing to a communicable illness. The use of Doxycycline in conjunction with basic metabolic enhancement therapy illuminates the opportunity for the Covid 19 virus to infect a human host extensively if at all. The use of doxycycline in those already infected with Covid 19 along with mucus reducing, superinfection mitigating and pneumonia reducing agents mitigates Covid 19 to progressing to SARS. An enhanced metabolic profile maximizes results.

### DETAILED DESCRIPTION OF THE INVENTION

Patient ingests 100 mg of Doxycycline daily which would allow for asymptomatic cases of Covid where the patient would not be able to progress to SARS and would be unable to spread the disease to others. Doxycycline 100 mg daily with metabolic enhancement package which would prevent as a whole a patient from contracting Covid 19. Doxycycline started in those with symptomatic 100 mg twice a day with mucus reducing agents to stop the disease from progressing to SARS. The Doxycycline is used as an ionophore to interrupt viral replication, as an anti-inflammatory as mucus build up causes morbidity and mortality. Doxycycline is also used as mitigation in the virus establishing replication ability via a symbiotic relationship with bacteria either symbiotic or pathological to humans.

### SPECIFICATION

Patient ingests 100 mg of Doxycycline daily which would allow for the mitigation of asymptomatic cases of Covid 19 or coronaviruses where the patient would not be able to progress to SARS and would be unable to spread the disease to others. Doxycycline 100 mg daily with metabolic enhancement package which would prevent as a whole a patient from contracting Covid 19. Metabolic enhancement package which would prevent as a whole a patient from contracting Covid 19. Doxycycline started in those with symptomatic 100 mg twice a day with mucus reducing agents to stop the disease from progressing to SARS. Supportive care medications used in conjunction with doxycycline to maximize benefits.

1. Cyclines or Doxycycline taken via ingestion in a range of 20 mg daily to 400 mg daily to prevent Covid 19.

2. Cyclins or Doxycycline taken in a range of 20 mg daily to 400 mg daily to prevent Covid 19 from progressing to SARS.

3. Metabolic immunity profile for the prevention of Covid 19 or to hinder the progression of Covid 19 to SARS like illness used in conjunction with cyclins or independently of cyclines; FISH OIL—BID EPA—1900 mg; DHA—750 mg; VIT—BID tablets combination a. Vit A 900 mg b. Vit C 500 mg in multivitamin tab c. Vit E 100 mg d. Zinc 120 mg e. Selenium 150 mg f. Copper 5 mg g. Lutein 6 mg CoQ-10-200 mg BID TIMOLOL 0.05 mg % diluted in 1 DROP in JUICE BID METFORMIN—One-tab PO BID Aspirin—81 mg po BID VIT-D—5000 IU BID VIT C—1000 mg BID separate from multivitamin tablets CITRIC ACID—½ tsp mixed in juice bid NIACIN—500 mg or 1000 mg if tolerated PO BID RIBOFLAVIN—100 MG BID ASTAXANTHIN 10 mg bid LACTOFERRIN 250 mg bid CURCUMIN 250 mg bid QUERCETIN 100 mg bid RESVERATROL 100 mg bid SULFORAPHANE 15 mg bid GLUCOSAMINE/CHONDROITIN—2 CAP PO BID

4. Cyclines or Doxycycline used as an ionophore to treat Covid 19 by allowing elements or trace elements into the cells to mitigate viral replication and activity.

5. Cyclines or Doxycycline used as an anti-inflammatory to treat Covid 19.

6. Cyclines or Doxycycline uses as an inhibitor of the virus using human symbiotic or pathological bacteria to replicate or to infect humans as a host.

7. Cyclines or Doxycycline uses independently as a decongestant or to hinder mucus build up.

8. Cyclines or Doxycycline used in conjunction with either, a combination of, or all of albuterol, ipratropium bromide, decongesting alcohols, guaifenesin, spironolactone, metronidazole, macrolides or other antibiotics to inhibit progression to SARS.

9. Cyclines or Doxyclines antiviral effects on coronavirus or covid 19 via anti-parisitic type of activity.

10. Cyclines or Doxyclines as an antiviral by interfering with the progress of the illness to SARS via the inhibition of the phosphorylation of the nucleocapsid protein of the coronavirus.

11. Cyclines or Doxyclines as an antiviral by interfering with the progress of the illness to SARS vie the inhibition of the process of translocation as it relates to the coronavirus

12. Cyclines or Doxycyclines used in conjunction with metronidazole or flagyl in cases where there is thick sticky mucus in the lungs where it is more likely for the development of bacterial injections.

13. Spironolactone, an aldosterone antagonist as an adjunct or independently by downregulating ACE2 receptor activity in connection or independently of a cycline.
